(19) United States
(12) Patent Application Publication
(10) Pub. No.: US 2022/0211008 A1
(43) Pub. Date: Jul. 7, 2022

(54) GENERATING PET IMAGE TRAINING DATA BASED ON SOURCE IMAGES

(71) Applicant: Société des Produits Nestlé S.A., Vevey (CH)

(72) Inventors: Everett Hullverson, Kirkwood, MO (US); Brian Michael ZANGHI, Ballwin, MO (US); Kyu S Cho, Ballwin, MO (US); Xiao Yang, Chesterfield, MO (US); Tonya Sue Ehlmann, St Peters, MO (US); James Clesie Moore, St. Louis, MO (US)

(21) Appl. No.: 17/557,212

(22) Filed: Dec. 21, 2021

Related U.S. Application Data
(60) Provisional application No. 63/134,756, filed on Jan. 7, 2021.

(51) Int. Cl. A01K 29/00; G06V 40/10; G06V 10/82; G06T 7/73

(52) U.S. Cl. CPC A01K 29/005; G06V 40/10; G06T 2207/20084; G06T 7/75; G06T 2207/20081; G06V 10/82

(57) ABSTRACT

A method can include receiving a first image depicting a first dog and identifying, with a first model, a first breed for the first dog based on the first image. The method may further include determining, with a second model, a first body condition for the first dog based on the first image; generating, with a third model, a second image depicting the first dog with a second body condition different from the first body condition. The method may also include labeling the first image with indications of the breed and the first body condition, labeling the second image with indications of the breed and the second body condition, and training the second model using the first and second images.

### CROSS REFERENCE TO RELATED APPLICATION

This application claims priority to U.S. Provisional Application Ser. No. 63/134,756 filed Jan. 7, 2021, the disclosure of which is incorporated in its entirety herein by this reference.

### BACKGROUND

Different pets may have different body conditions depending on various physical and life-stage factors such as body size, body shape, muscle structure, age, and weight of the pet for any given breed. Knowing the body condition of a pet may be helpful in analyzing the health of the pet and in performing other health-related tasks for the pet, such as preparing pet food compositions and deriving feeding recommendations based on the pet's body condition.

### SUMMARY

The present disclosure presents new and innovative methods. In some embodiments, the method can include: receiving a first image depicting a first dog; identifying, with a first model, a first breed for the first dog based on the first image; determining, with a second model, a first body condition for the first dog based on the first image; generating, with a third model, a second image depicting the first dog with a second body condition different from the first body condition; labeling the first image with indications of the breed and the first body condition; labeling the second image with indications of the breed and the second body condition; and training the second model using the first and second images.

The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the disclosed subject matter.

### BRIEF DESCRIPTION OF THE FIGURES

FIG. 1 illustrates a system for analyzing the body condition of a dog according to an exemplary embodiment of the present disclosure.

FIG. 2 shows morphometric measurements that may be used to determine a dog's body condition according to an exemplary embodiment of the present disclosure.

FIG. 3 illustrates a model taxonomy according to an exemplary embodiment of the present disclosure.

FIGS. 4A-4C illustrate images of a dog according to an exemplary embodiment of the present disclosure.

FIG. 5 illustrates a method for training a body condition model according to an exemplary embodiment of the present disclosure.

FIG. 6 illustrates a computing system according to an exemplary embodiment of the present disclosure.

### DETAILED DESCRIPTION OF EXAMPLE EMBODIMENTS

As used in this disclosure and the appended claims, the singular forms “a,” “an” and “the” include plural referents unless the context clearly dictates otherwise. Thus, for example, reference to “a dog” or “the dog” includes two or more dogs.

The words “comprise,” “comprises” and “comprising” are to be interpreted inclusively rather than exclusively. Likewise, the terms “include,” “including” and “or” should all be construed to be inclusive, unless such a construction is clearly prohibited from the context.

However, the compositions disclosed herein may lack any element that is not specifically disclosed. Thus, a disclosure of an embodiment using the term “comprising” includes a disclosure of embodiments “consisting essentially of” and “consisting of” the components identified. Similarly, the methods disclosed herein may lack any step that is not specifically disclosed herein. Thus, a disclosure of an embodiment using the term “comprising” includes a disclosure of embodiments “consisting essentially of” and “consisting of” the steps identified.

The term “and/or” used in the context of “X and/or Y” should be interpreted as “X,” or “Y,” or “X and Y.” Where used herein, the terms “example” and “such as,” particularly when followed by a listing of terms, are merely exemplary and illustrative and should not be deemed to be exclusive or comprehensive. Any example or embodiment disclosed herein can be combined with any other example or embodiment disclosed herein unless explicitly stated otherwise.

Numerical adjectives, such as “first” and “second,” are merely used to distinguish components. These numerical adjectives do not imply the presence of other components, a relative positioning, or any chronological implementation. In this regard, the presence of a “second food composition” does not imply that a “first food composition” is necessarily present. Further in this regard, a “second food composition” can be used before, after, or simultaneously with any “first food composition.”

All percentages expressed herein are by weight of the total weight of the composition unless expressed otherwise. As used herein, “about” and “approximately” are understood to refer to numbers in a range of numerals, for example the range of −10% to +10% of the referenced number, preferably within −5% to +5% of the referenced number, more preferably within −1% to +1% of the referenced number, most preferably within −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

The term “dog” means all canine animals, non-limiting examples of which include pet dogs, working dogs, show dogs, guard dogs, scent hounds, and retrievers. The term “puppy” means an animal of any age in the juvenile growth and development stage. For example, a dog that is a “puppy” typically has an age up to about 1 year. The term “adult” means an animal of an age after the completion of the juvenile growth and development stage until development of an increased risk of age-related disease. For example, a dog that is an “adult” typically has an age from about 1 year to about 7 years. The term “senior” means an animal of an age having an increased risk for age-related disease but may or may not have obvious physical or behavioral characteristics of aging. For example, a dog that is a “senior” typically has an age from about 7 years to about 11 years. The term “geriatric” means an animal showing outward signs of aging. For example, a dog that is “geriatric” typically has an age of about 11 years or more.

The term “small dog” means a dog that weighs less than 10 kg. The term “miniature dog” means a dog that weighs less than 5 kg, and miniature dogs are encompassed by the term “small dog.” The term “medium dog” means a dog that weighs between 10 and 25 kg. The term “large dog” means a dog that weighs between 25 and 40 kg. The term “giant dog” means a dog that weighs more than 40 kg.

The term “food composition” means any composition suitable for consumption by a dog, including, but not limited to, dry, wet, semi-moist, moist, and liquid food compositions.

The methods and devices and other advances disclosed herein are not limited to particular methodologies, protocols, and reagents because, as the skilled artisan will appreciate, they may vary. Further, the terminology used herein is for the purpose of describing particular embodiments only and does not limit the scope of that which is disclosed or claimed.

Unless defined otherwise, all technical and scientific terms, terms of art, and acronyms used herein have the meanings commonly understood by one of ordinary skill in the art in the field(s) of the present disclosure or in the field(s) where the term is used. Although any compositions, methods, articles of manufacture, or other means or materials similar or equivalent to those described herein can be used, the preferred devices, methods, articles of manufacture, or other means or materials are described herein.

Additionally, the embodiments discussed herein focus on dogs and canine animals. However, it should be understood that similar techniques may be used to generate images of other types of pets or animals. For example, similar techniques may be used for cats or feline animals, horses, cows, pigs, and the like.

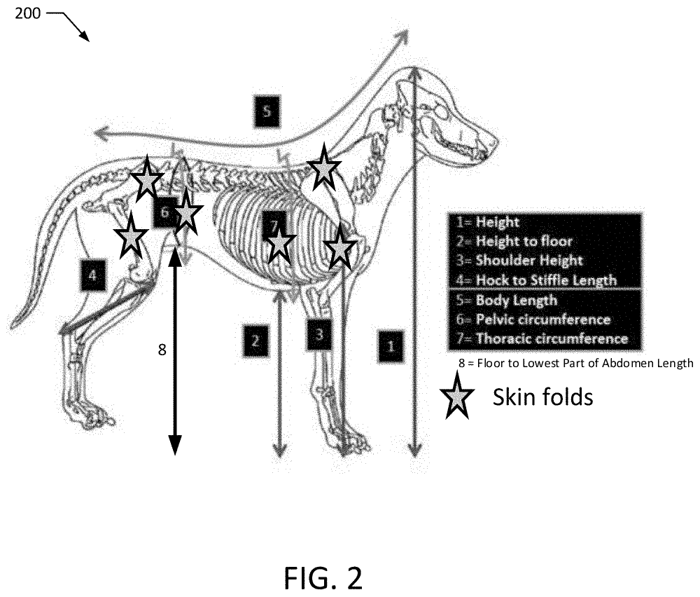

The body condition of a dog may be determined by measurements taken of the dog. For example, weight and morphometric measurements (such as those discussed below in connection with FIG. 2) may be taken of the dog and may be used to determine a body condition for the dog. U.S. patent application Ser. No. 15/229,367 (entirely incorporated herein by reference) describes how such measurements may be used to determine a body condition for a dog. However, such techniques may be difficult to scale, as each dog needs to be measured by a veterinarian or other domain expert or by a pet owner. Further, pet owners may not properly measure their dogs, resulting in inaccurate body conditions for the dog.

In certain instances, images of the dog may be used to determine the body condition of the dog. For example, a domain expert (e.g., a veterinarian) may receive and analyze images of dogs and may determine a breed and body condition for each dog. However, such systems are limited by the rate at which domain experts can receive and analyze images, which may increase cost and processing time to determine body conditions for the dogs. Accordingly, there exists a need to analyze and determine body conditions for dogs on an automated basis.

One solution to this problem is to use machine learning models to determine body conditions of dogs based on images of the dogs. For example, one or more machine learning models may be used to detect a breed of a dog depicted in an image and to determine a body condition of the dog based on the detected breed. In particular, a first machine learning model may be used to detect the breed of the dog. Based on the breed, a second machine learning model may be selected and used to determine the body condition. Utilizing separate models to detect body condition for different breeds may improve accuracy, as different breeds may have different physical characteristics with different body conditions. However, utilizing separate machine learning models for different breeds requires the creation and training of many more models than using a single machine learning model to determine body condition. Accordingly, additional training data may be required to accurately train models for different types of breeds. To provide additional training data, existing images of dogs may be transformed to create new images of dogs in different body conditions. For example, an image of a dog that is overweight may be transformed into images of the dog at an ideal weight and/or at an obese weight. In particular, the images may be transformed by one or more image transformation models.

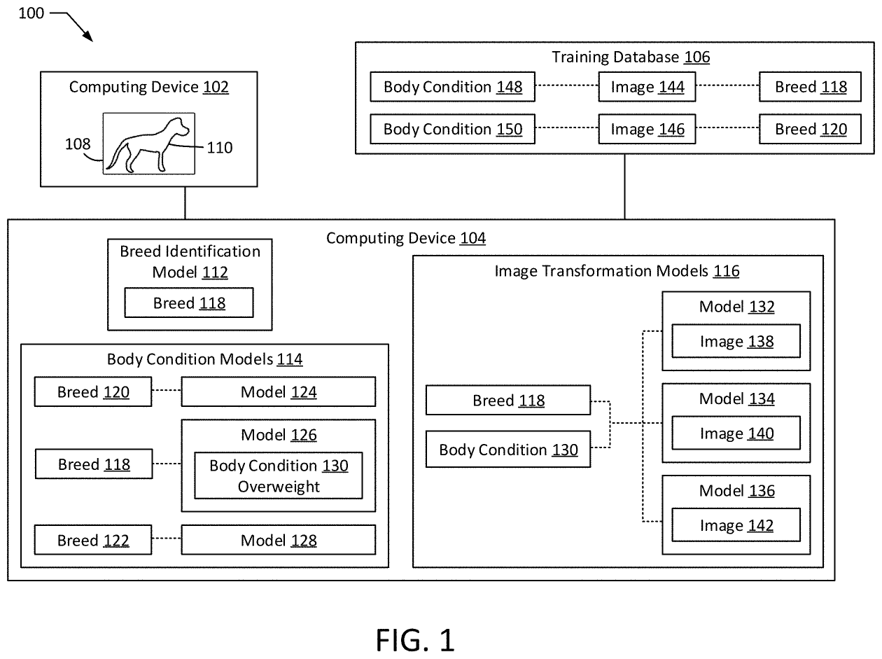

FIG. 1 illustrates a system 100 for training body condition models according to an exemplary embodiment of the present disclosure. The system 100 may be configured to receive and analyze images of dogs to determine body conditions of the dogs. The body conditions of the dogs may further be used to generate or recommend particular pet food compositions for the dogs. The system 100 may also be configured to transform received images of dogs with a particular body condition into images of the dogs with different body conditions.

The system 100 includes computing devices 102, 104 and a training database 106. The computing device 104 may be configured to receive and analyze images from other computing devices. For example, the computing device 104 may receive and analyze images 108 from computing devices 102 associated with pet owners, such as smart phones, tablet computers, laptops, personal computers, and the like. In one specific example, the computing device 104 may receive an image 108 from a computing device 102 associated with a pet owner that depicts a dog 110.

The computing device 104 may analyze the image 108 determine a breed 118 of the dog 110. For example, the computing device 104 may include a breed identification model 112 trained to analyze the image 108 to determine the breed 118 of the dog 110. The breed identification model 112 may be implemented by one or more machine learning models, including one or more supervised learning models, unsupervised learning models, or other types of machine learning models. For example, the breed identification model 112 may be implemented as one or more of a neural network, a decision tree model, a support vector machine, and a Bayesian network. In certain implementations, the breed identification model 112 may be implemented as a deep convolutional neural network (deep CNN) with transfer learning and ensemble learning.

Based on the identified breed 118, the computing device 104 may determine a body condition 130 of the dog 110. Body conditions 130 may be determined to represent a physical health status for the dog 110 based on a size and/or weight of the dog 110. For example, the computing device 104 includes multiple body condition models 114. For example, body conditions may include may include an underweight body condition for dogs that weigh less than they should, an ideal body condition for dogs that weigh what they should, an overweight body condition who weigh more than they should, and an obese body condition who weigh much more than they should. In additional or alternative implementations, the body conditions 130 may be determined as a score (e.g., a score from 1-10) ranging from underweight to obese.

The body condition models 114 include multiple models 124, 126, 128 associated with different breeds 118, 120, 122 of dogs. As will be appreciated by those skilled in the art, dogs of different breeds may present different physical characteristics with different body conditions. For example, greyhounds may typically be lean and may accordingly have a lower ideal body weight, smaller body measurement proportions, and/or a lower body fat percentage to be considered an ideal body condition. By contrast, St. Bernard or Alaskan Husky dogs may typically be heavier, have larger body measurement proportions, have a greater body fat percentage, and/or may have a higher ideal body weight that is considered an ideal body condition. If a single body condition model were used to determine a body condition for greyhounds and St. Bernard dogs, the model may improperly classify a greyhound with an ideal body weight as having an underweight body condition. Such a model may also improperly classify a St. Bernard dog with an ideal body weight as having an overweight body condition.

Accordingly, the computing device 104 may use separate models 124, 126, 128 to detect body conditions 130 for different breeds. In particular, the models 124, 126, 128 correspond to breeds 118, 120, 122, which may represent one or more of beagles, bulldogs, Chihuahuas, German shepherds, Labrador retrievers, golden retrievers, Corgis, Greyhounds, St. Bernard dogs, and any other breed of dog or mixed breed of dog. Based on the breed 118 of the dog 110, the computing device 104 may use a corresponding model 126 to identify a body condition of the dog 110 based on the image 108. For example, based on the image 108, the model 126 may identify a body condition 130 of overweight for the dog 110.

The body condition models 114 may be implemented by one or more machine learning models, including one or more supervised learning models, unsupervised learning models, or other types of machine learning models. In certain implementations, the body condition models 114 may be implemented as a neural network, such as neural networks with dense mapping, convolutional neural networks, recurrent neural networks, and the like. The body condition models 114 may be trained to determine body conditions for the corresponding breeds 118, 120, 122. For example, the models 124, 126, 128 may be trained to determine body conditions for dogs 110 depicted in received images 108 based on one or more features include a size of the dog 110 within the image, morphometric measurements of the dog 110 (such as those discussed below in connection with FIG. 2), a mask of the dog 110 within the image 108 (e.g., an outline or area occupied by the dog 110 within the image 108), the locations of one or more user-placed markers on the dog 110 within the image 108, and a predicted weight of the dog 110. Based on the present disclosure, one skilled in the art may understand that additional or alternative features may be utilized by the models 124, 126, 128 to predict a body condition 130 for dogs 110. Furthermore, one skilled in the art may understand that the models 124, 126, 128 may be implemented by additional or alternative types of models. All such features and methods are hereby contemplated within the scope of the present disclosure.

In the above-discussed implementations, each model 124, 126, 128 is associated with a single breed 118, 120, 122. However, in additional or alternative implementations, at least a subset of the models 124, 126, 128 may correspond to more than one breed 118, 120, 122. For example, a single model may correspond to all retriever breeds (e.g., Labrador retrievers and Golden retrievers). In certain instances, the same model may be used to identify body conditions for similar sizes of dogs (e.g., toy dogs, small dogs, medium dogs, large dogs, extra large dogs, giant dogs). For example, a single model may be used to identify body conditions for medium-sized dogs (e.g., cocker spaniels, basset hounds, beagles, Boston terriers). In further instances, models may be used to identify body conditions for similar types of dogs. For example, a single model may be used to determine the body condition of small, athletic dog breeds (e.g., Chihuahuas, miniature pinschers).

The computing device 104 may transform received images 108 into additional images using one or more image transformation models 116. For example, the image transformation models 116 may be used to generate additional images of the dog 110 based on the image 108. In particular, the image transformation models 116 may be used to generate images 138, 140, 142 that depict the dog 110 with body conditions other than the body condition 130 identified by the body condition models 114. For example, the image transformation models 116 include models 132, 134, 136 that correspond to the breed 118 and the body condition 130. In particular, each of the models 132, 134, 136 may be configured to transform images of dogs of the breed 118 from the body condition 130 to other body conditions. As a specific example, where the body condition 130 is overweight (as depicted), the model 132 may be configured to transform the image 108 into an image 138 of the dog 110 with an underweight body condition.

The model 134 may be configured to transform the image 108 into an image 140 of the dog 110 with an ideal body condition. The model 136 may be configured to transform the image 108 into an image 142 of the dog 110 with an obese body condition.

One or more of the breed identification model 112, the body condition models 114, and the image transformation models 116 may be trained based on images of dogs with identified breeds and/or body conditions. As a specific example, the system 100 includes a training database 106, which may store data used to train the breed identification model 112, the body condition models 114, and/or the image transformation models 116. In particular, the training database 106 stores images 144, 146 in association with identifiers of breeds 118, 120 and body conditions 148, 150. To train the breed identification model 112, the computing device 104 may analyze one or more of the images 144, 146 with the breed identification model 112 to predict a breed of the dogs depicted in the images 144, 146. The breed predicted by the breed identification model 112 may be compared to the corresponding breeds 118, 120 in the training database 106. Parameters of the breed identification model 112 may be updated based on whether the breed identification model 112 correctly identified the breeds of the dogs within the images 144, 146. To train the body condition models 114, the computing device 104 may analyze one or more of the images 144, 146 to predict a body condition for the dogs depicted in the images 144, 146. The body condition predicted may be compared to the body conditions 148, 150 associated with the images 144,146. Parameters of the body condition models 114 may be updated based on whether the body condition models 114 correctly identify the body condition for the dogs depicted in the images 144, 146. One or more of the breed identification model 112 and the body condition models 114 may include weights (e.g., priorities) for different features and combinations of features of images. Updating the parameters of the breed identification model 112 and the body condition models 114 may include updating one or more of the analyzed features, the weights assigned to different features, and/or combinations of features.

Accurately training the breed identification model 112 and each of the models 124, 126, 128 included within the body condition models 114 may accordingly require a large number of images within the training database 106. For example, training a model 124, 126, 128 associated with a particular breed may require 400-600 images of the particular breed of dog in each of the body conditions to be classified. Accordingly, accurately training a model 124, 126, 128 to detect four body conditions (underweight, ideal, overweight, obese) may require 1600-2400 images of dogs for each breed 118, 120, 122. To increase the number of available images in the training database 106, the images 138, 140, 142 generated by the image transformation models 116 may be stored for future use in the training database 106. For example, the images 138, 140, 142 may be used to train the model 126 corresponding to the breed 118. In particular, the images 138, 140, 142 may be stored in combination with tags or other indications of the breed 118 and the body condition depicted in the images 138, 140, 142, similar to the indications of the body conditions 148, 150 and breeds 118, 120 for the images 144, 146. For example, the image 138 may be stored in association with identifiers of the breed 118 and an underweight body condition. The image 140 may be stored in association with identifiers of the breed 118 and an ideal body condition. The image 142 may be stored in association with identifiers of the breed 118 and an obese body condition. In further instances, the image 108 may also be stored in connection with an identifier of the breed 118 and the body condition 130. In certain instances, prior to storing the images 108, 138, 140, 142 in the training database 106, one or more quality analyses may be performed. For example, the images 138, 140, 142, or a subset thereof, may be analyzed by domain experts to ensure that the images 138, 140, 142 accurately depict dogs with the corresponding body conditions.

In certain instances, the breed 118 and/or the body condition 130 may not be determined using the breed identification model 112 and the body condition models 114. For example, the computing device 104 may receive indications of the breed 118 and/or the body condition 130 from a user (e.g., a user of the computing device 102), such as an owner of the dog 110, a veterinarian, a domain expert, and the like.

One or more of the computing devices 102, 104 and the training database 106 may be implemented by a computing system. For example, although not depicted, one or more of the computing devices 102, 104 and the training database 106 may contain a processor and a memory that implement at least one operational feature. For example, the memory may contain instructions which, when executed by the processor, cause the processor to implement at least one operational feature of the computing devices 102, 104 and/or the training database 106.

FIG. 2 shows morphometric measurements 200 that may be used to determine a dog's body condition according to an exemplary embodiment of the present disclosure. For example, in certain instances, the breed identification model 112 and/or one or more of the body condition models 114 may extract or determine one or more morphometric measurements 200 of the dog 110 when identifying the breed 118 of the dog 110 and/or when determining the body condition 130 of a dog 110. As depicted, the morphometric measurements 200 include height sternum to floor, shoulder height, body length, pelvic circumference, thoracic circumference, and floor to lowest part of abdomen length. The height sternum to floor may be defined as the distance from the floor to the lowest point of the sternum. The shoulder height may be defined as the distance from the floor to the tip of the acromion. Body length may be defined as the distance between the occiput and the base of the tail. Thoracic circumference may be defined as the circumference of the widest point of the thorax. Pelvic circumference may be defined as the circumference of the thinnest point of the thorax. The floor to lowest part of abdomen length may be defined as the distance from the floor to the lowest point of the abdomen (e.g., at or around the pelvis).

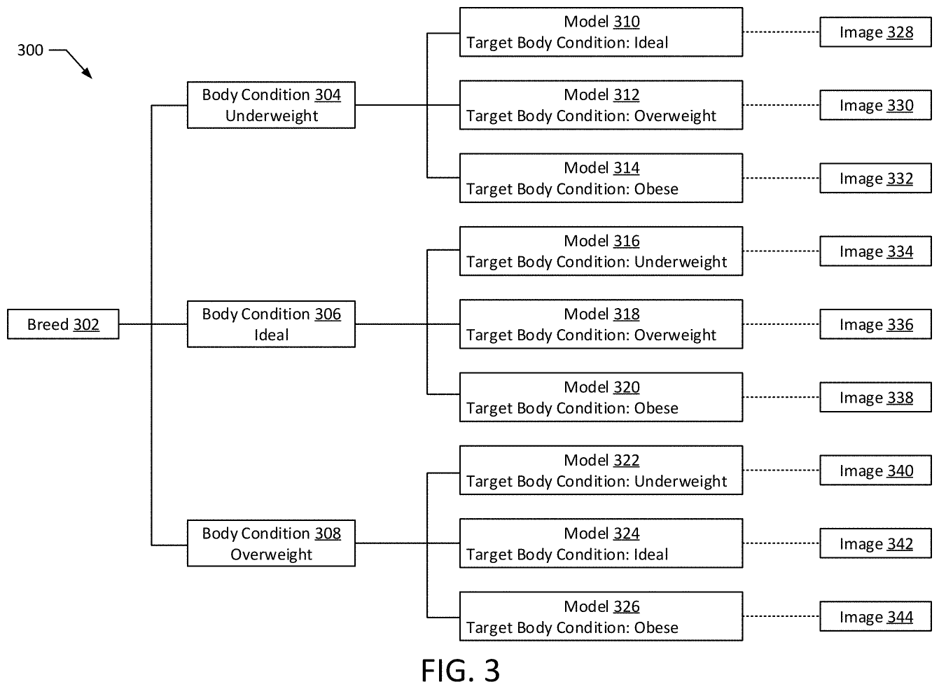

FIG. 3 illustrates a model taxonomy 300 according to an exemplary embodiment of the present disclosure. The model taxonomy 300 may depict image transformation models used to generate images for training body condition models. For example, the model taxonomy 300 may be an exemplary implementation of a subset of the image transformation models 116 used by the computing device 104. In particular, the model taxonomy 300 includes models 310, 312, 314, 316, 318, 320, 322, 324, 326 for a breed 302 of dog. The models 310, 312, 314, 316, 318, 320, 322, 324, 326 may have a different corresponding initial body condition and target body condition. For example, each model 310, 312, 314, 316, 318, 320, 322, 324, 326 corresponds to a body condition 304, 306, 308, which may represent the initial body condition and includes a target body condition. The models 310, 312, 314, 316, 318, 320, 322, 324, 326 may be configured to generate images 328, 330, 332, 334, 336, 338, 340, 342, 344 that depict dogs of the breed 302 with the target body condition based on images of dogs with the corresponding initial body condition 304, 306, 308.

A subset of the models 310, 312, 314, 316, 318, 320, 322, 324, 326 may be used to generate additional images based on a received image. The subset of the models 310, 312, 314, 316, 318, 320, 322, 324, 326 may be selected based on a breed 302 and/or the body condition 304, 306 of the dog depicted in the received image. As explained above, the breed 302 may be determined by a breed identification model and/or may be indicated by a user (e.g., an owner, a veterinarian, a domain expert). The body condition 304, 306, 308 may be determined by a body condition model and/or may be received from a user. The subset of the models 310, 312, 314, 316, 318, 320, 322, 324, 326 may then be selected as the models 310, 312, 314, 316, 318, 320, 322, 324, 326 corresponding to the body condition 304, 306, 308 and the breed 302. As a specific example, if a dog depicted in a received image has a body condition 306 of overweight, the models 322, 324, 326 may be used to transform the received image into images 340, 342, 344 of the dog with underweight, ideal, and obese body conditions.

FIG. 3 depicts models corresponding to three body conditions: underweight, ideal, and overweight. It should be understood that additional or alternative implementations may include models corresponding to other body conditions, such as obese. Furthermore, other types of body conditions may be used in connection with the techniques and systems discussed herein.

For example, body conditions may include a score from 1-9 and each score may include multiple corresponding models for the remaining body conditions (e.g., the 8 remaining body conditions). Further classifications of body conditions may be apparent to one skilled in the art in light of the present disclosure. All such techniques are considered within the scope of the present disclosure.

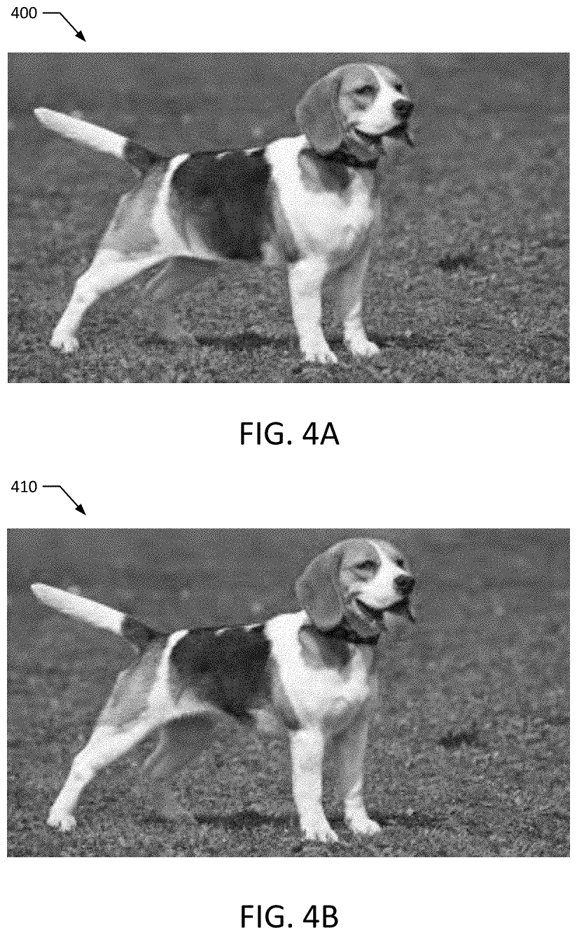

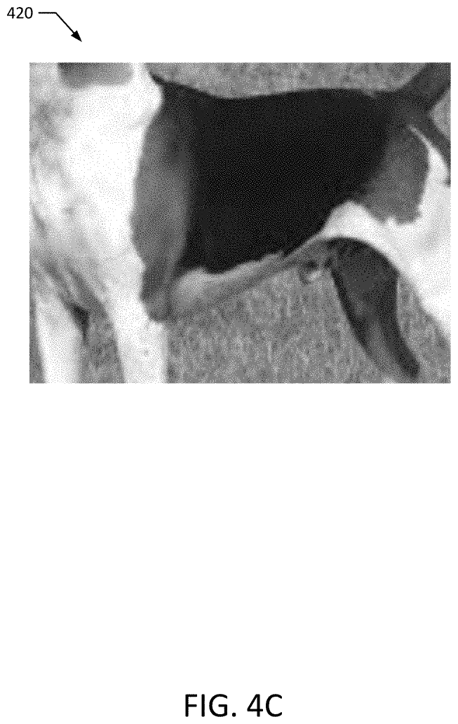

In one particular instance, FIGS. 4A-4C illustrate images 400, 410, 420 of dogs according to an exemplary embodiment of the present disclosure. In particular, the image 400 may be an exemplary received image of a beagle dog with an overweight body condition. The image 410 depicts the beagle dog with an ideal body condition, which may be generated by the model 324 based on the image 400. To generate images 328, 330, 332, 334, 336, 338, 340, 342, 410, the image transformation models 116 (e.g., the models 124, 126, 128, 310, 312, 314, 316, 318, 320, 322, 324, 326) may utilize one or more example images of dogs that are the same breed 302 as the dog depicted in a received image. For example, the image 420 depicts a beagle dog with an ideal body condition. The image transformation models 116 may be configured to utilize one or more style transfer techniques to transfer a style of the example image 420 to a received image 400. As a specific example, one or more of the image transformation models 116 may be implemented by one or more neural networks, such as a generative-adversarial network model trained to transfer the style of example images to received images. As another example, one or more of the image transformation models 116 may be implemented as one or more deep learning networks, such as convolutional neural networks (CNNs).

In still further implementations, received images 108, 400 may be processed before analysis by the models 112, 114, 116. For example, the images may be cropped, rotated, or otherwise processed to improve the accuracy of processing by the models 112, 114, 116. In one specific example, cropping received images 108, 400 to focus on one or more subjects of the images 108, 400 may improve the accuracy of one or more breed identification models, body condition models 114, and/or image transformation models. In certain such instances, received images 108, 400 may be cropped by a You Only Look Once (YOLO) real-time object detection model that is trained to detect certain types of objects, such as pets (e.g., dogs 110, cats, or other pets), within received images 108. The YOLO model may output a bounding box that surrounds the detected objects and cropped images may be extracted from received images 108 based on the bounding boxes. For example, a received image may depict two dogs and the YOLO model may output two bounding boxes: a first bounding box that surrounds a first dog and a second bounding box that surrounds a second dog. The YOLO model may extract separate cropped images defined by each of the first and second bounding boxes (e.g., that contain at least a subset of the pixels contained within the first and second bounding boxes) and may continue processing each of the cropped images separately with the models 112, 114, 116, as described further herein.

In still further implementations, the cropped images may be processed along with original, received images 108, 400. For example, to accurately detect a breed 118, the breed identification model 112 may process two separate images: a first, received image 108 depicting a dog 110 and a second, cropped image of the dog generated based on the YOLO model. When detecting a breed 118 based on each of the images, the breed identification model 112 may output a confidence measure of the detected breed 118. In such instances, the breed 118 for the dog may be identified as the breed detected by the breed identification model 112 based on the image with the highest corresponding confidence measure. Similar techniques may be used for the body condition models 114 and/or the image transformation models 116 as well. Utilizing both images in this way may maximize the accuracy of pet image processing and breed detection.

Furthermore, in certain instances, before processing by the models 112, 114, 116, received images 108, 400 may be analyzed to determine that they depict a side view of the pet, which may be necessary to accurately determine the body condition and/or to generate transformed images 138, 140, 142, 410 based on received images 108, 400. In implementations 17814 that also crop received images using a YOLO model, this analysis may be performed before cropping the images with the YOLO model and/or after cropping the images with the YOLO model. In one instance, received images 400 may be analyzed by an image classifier model (e.g., machine learning model such as a CNN) trained to detect side view images of animals (e.g., dogs 110, cats, or other pets). If the image classifier model detects that a received image 108, 400 depicts a side view of at least one pet, subsequent processing may continue with the models 112, 114, 116 and/or the YOLO model. If the image classifier model detects that a received image 108, 400 does not depict a side view of at least one pet, the image 108, 400 may not be processed, and an error may be presented to a user (e.g., a user who uploaded the image 108, 400) that requests the user to upload a side view image of their pet.

Similarly, additional techniques (e.g., additional machine learning models) may be used to confirm other characteristics of received images before further processing by the models 112, 114, 116. For example, the models 112, 114 may not be trained to detect breeds or determine body conditions for puppies or other juvenile pets. In such instances, a model may analyze received images 108, 400 to determine whether the images 108, 400 depict puppies or other juvenile animals. If so, a message may be presented to a user indicating that the system cannot process images for juvenile animals. Additionally or alternatively, the computing device 104 may select a different set of breed identification models, body condition models, and image transformation models that are trained to work with juvenile animals. If a juvenile animal is not detected, processing may continue as discussed herein.

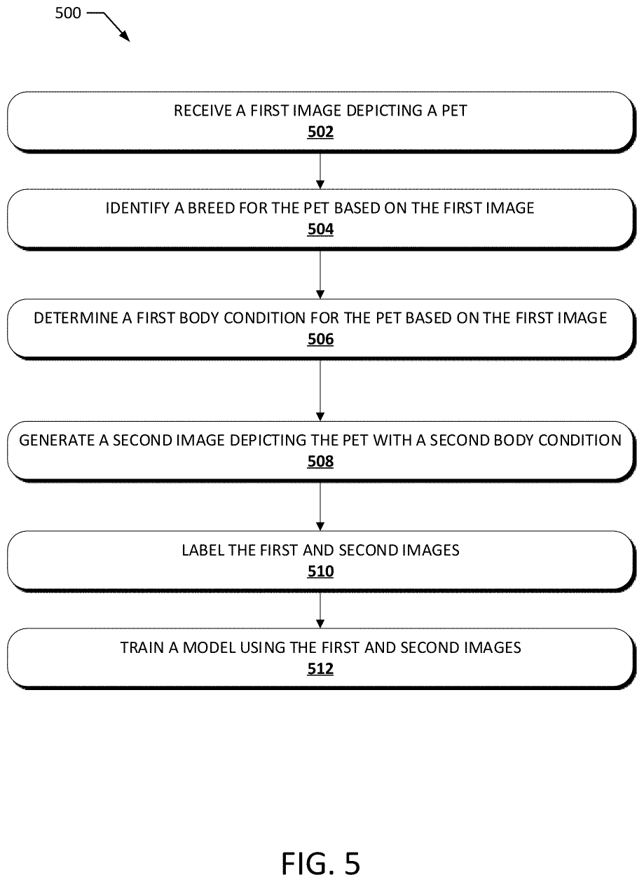

FIG. 5 illustrates a method 500 for training a body condition model according to an exemplary embodiment of the present disclosure. The method 500 may be implemented on a computing system, such as the system 100. For example, the method 500 may be implemented by the computing device 102, the computing device 104, and/or the training database 106.The method 500 may also be implemented by a set of instructions stored on a computer-readable medium that, when executed by a processor, cause the computer system to perform the method 500. For example, all or part of the method 500 may be implemented by a processor and/or a memory of the computing device 102, the computing device 104, and/or the training database 106. Although the examples below are described with reference to the flowchart illustrated in FIG. 5, many other methods of performing the acts associated with FIG. 5 may be used. For example, the order of some of the blocks may be changed, certain blocks may be combined with other blocks, one or more of the blocks may be repeated, and some of the blocks described may be optional.

The method 500 may begin with receiving a first image depicting a pet (block 502). For example, a first image 108 may be received of a pet, such as a dog 110. The first image 108 may be received from computing device 102. For example, the first image 108 may be received from a computing device 102 associated with, e.g., an owner of the pet, a veterinarian associated with the pet, and/or another user. The first image 108 may depict a side view of the pet. In certain instances, the first image 108 may be captured by a camera connected to the computing device 102. For example, the computing device 102 may be a smart phone, tablet, or other computing device equipped with a camera and the image 108 may be captured by the camera.

A breed may be identified for the pet based on the first image (block 504). For example, the computing device 104 may identify a breed 118 of the pet based on the first image 108. In certain instances, the breed 118 may be identified using a breed identification model 112, as explained above. In additional or alternative implementations, the breed may be provided with the first image 108. For example, the breed 118 may be received along with the first image 108 from the computing device 102. In certain instances, the breed may be identified by a domain expert (e.g., a veterinarian or dog breed expert).

A first body condition for the pet may be determined based on the first image (block 506). For example, a first body condition 130 may be determined for the pet based on the first image 108 by the computing device 104. In certain instances, the first body condition 130 may be determined based on a machine learning model, such as one or more body condition models 114. In particular, the first body condition 130 may be determined by a model 126 that corresponds to the breed 118 identified a block 504. As explained above, the model 126 may be selected from among a plurality of body condition models 114, where each of the models 124, 126, 128 correspond to one or more breeds 120, 118, 122 of the pet.

A second image may be generated depicting the pet with a second body condition (block 508). For example, a second image 138, 140, 142 of the pet may be generated. The second image may be generated by one or more machine learning models, such as image transformation models 116. In particular, the computing device 104 may include one or more models 132, 134, 136 for transforming images of pets of the breed 118 with the first body condition 130. In certain implementations, multiple models 132, 134, 136 may correspond to the breed 118 and the body condition 130. In such instances, multiple images 138, 140, 142 may be generated of the pet 110 with multiple different body conditions. As further explained in connection with FIG. 3, each of the models that correspond to the first body condition may have a particular target body condition, which may constitute the second body condition.

The first and second images may be labeled (block 510). For example, the first and second images 108, 138, 140, 142 may be labeled with indications of one or more of a type of pet, a breed of the pet, and the body conditions depicted in each of the images 108, 138, 140, 142. For example, metadata may be added to the image files for the first and second images 108, 138, 140, 142 that identifies (i) the type of pet (e.g., dog, cat, etc.), (ii) the breed 118 of pet, and (iii) the body condition depicted in the image 108, 138, 140, 142. Once labeled, the images 108, 138, 140, 142 and associated labels may be stored in a training database 106.

The first and second images 108, 138, 140, 142 may be used to train a model (block 512). For example, the first and second images 108, 138, 140, 142 may be used train one or more of a breed identification model, a pet type identification model, and/or a body condition model to identify body conditions for the breed 118. It should be understood that, in practice, the first and second images 108, 138, 140, 142 may be used to train other types of models, as may be readily apparent to one skilled in the art in light of the present disclosure.

In this way, the method 500 enables rapid generation of image training data for machine learning models that may be used to identify one or more aspects of an image of a pet. In this way, the method 500 and the techniques discussed herein may improve the accuracy of identified pet reads and body conditions. Such accuracy may enable automated generation of pet food formulations, proactive pet nutrition, feeding recommendations, pet wellness recommendations, age detection, and pet characterization tagging. Accordingly, these techniques may improve the overall quality of life and longevity for many types and breeds of pets.

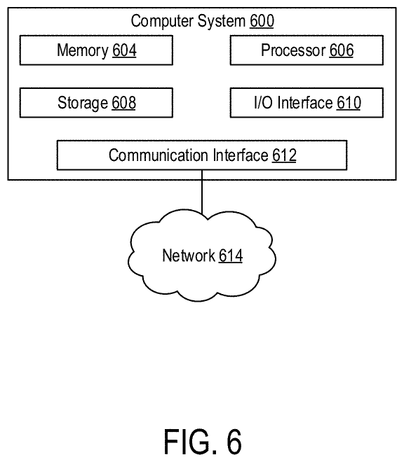

FIG. 6 illustrates a computing system 600 according to an exemplary embodiment of the present disclosure. The computing system 600 may be used to implement one or more of the computing devices 102, 104 and the training database 106. The computing system 600 may also be implemented to implement one or more techniques discussed herein, such as the method 500. In particular embodiments, one or more computer systems 600 provide the functionalities described or illustrated herein. In particular embodiments, software running on one or more computer systems 600 performs one or more steps of one or more methods described or illustrated herein or provides the functionalities described or illustrated herein. Particular embodiments include one or more portions of one or more computer systems 600. Herein, a reference to a computer system may encompass a computing device, and vice versa, where appropriate. Moreover, a reference to a computer system may encompass one or more computer systems, where appropriate.

This disclosure contemplates any suitable number of computer systems 600. This disclosure contemplates the computer system 600 taking any suitable physical form. As example and not by way of limitation, the computer system 600 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, the computer system 600 may include one or more computer systems 600; be unitary or distributed; span multiple locations; span multiple machines; span multiple data centers; or reside in a cloud, which may include one or more cloud components in one or more networks. Where appropriate, one or more computer systems 600 may perform without substantial spatial or temporal limitation one or more steps of one or more methods described or illustrated herein. As an example and not by way of limitation, one or more computer systems 600 may perform in real time or in batch mode one or more steps of one or more methods described or illustrated herein. One or more computer systems 600 may perform at different times or at different locations one or more steps of one or more methods described or illustrated herein, where appropriate.

In particular embodiments, computer system 600 includes a processor 606, memory 604, storage 608, an input/output (I/O) interface 610, and a communication interface 612. Although this disclosure describes and illustrates a particular computer system having a particular number of particular components in a particular arrangement, this disclosure contemplates any suitable computer system having any suitable number of any suitable components in any suitable arrangement.

In particular embodiments, the processor 606 includes hardware for executing instructions, such as those making up a computer program. As an example and not by way of limitation, to execute instructions, the processor 606 may retrieve (or fetch) the instructions from an internal register, an internal cache, memory 604, or storage 608; decode and execute the instructions; and then write one or more results to an internal register, internal cache, memory 604, or storage 608. In particular embodiments, the processor 606 may include one or more internal caches for data, instructions, or addresses. This disclosure contemplates the processor 606 including any suitable number of any suitable internal caches, where appropriate. As an example and not by way of limitation, the processor 606 may include one or more instruction caches, one or more data caches, and one or more translation lookaside buffers (TLBs). Instructions in the instruction caches may be copies of instructions in memory 604 or storage 608, and the instruction caches may speed up retrieval of those instructions by the processor 606. Data in the data caches may be copies of data in memory 604 or storage 608 that are to be operated on by computer instructions; the results of previous instructions executed by the processor 606 that are accessible to subsequent instructions or for writing to memory 604 or storage 608; or any other suitable data. The data caches may speed up read or write operations by the processor 606. The TLBs may speed up virtual-address translation for the processor 606. In particular embodiments, processor 606 may include one or more internal registers for data, instructions, or addresses. This disclosure contemplates the processor 606 including any suitable number of any suitable internal registers, where appropriate. Where appropriate, the processor 606 may include one or more arithmetic logic units (ALUs), be a multi-core processor, or include one or more processors 606. Although this disclosure describes and illustrates a particular processor, this disclosure contemplates any suitable processor.

In particular embodiments, the memory 604 includes main memory for storing instructions for the processor 606 to execute or data for processor 606 to operate on. As an example, and not by way of limitation, computer system 600 may load instructions from storage 608 or another source (such as another computer system 600) to the memory 604. The processor 606 may then load the instructions from the memory 604 to an internal register or internal cache. To execute the instructions, the processor 606 may retrieve the instructions from the internal register or internal cache and decode them. During or after execution of the instructions, the processor 606 may write one or more results (which may be intermediate or final results) to the internal register or internal cache. The processor 606 may then write one or more of those results to the memory 604. In particular embodiments, the processor 606 executes only instructions in one or more internal registers or internal caches or in memory 604 (as opposed to storage 608 or elsewhere) and operates only on data in one or more internal registers or internal caches or in memory 604 (as opposed to storage 608 or elsewhere). One or more memory buses (which may each include an address bus and a data bus) may couple the processor 606 to the memory 604. The bus may include one or more memory buses, as described in further detail below. In particular embodiments, one or more memory management units (MMUs) reside between the processor 606 and memory 604 and facilitate accesses to the memory 604 requested by the processor 606. In particular embodiments, the memory 604 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 604 may include one or more memories 604, where appropriate. Although this disclosure describes and illustrates particular memory implementations, this disclosure contemplates any suitable memory implementation.

In particular embodiments, the storage 608 includes mass storage for data or instructions. As an example and not by way of limitation, the storage 608 may include a hard disk drive (HDD), a floppy disk drive, flash memory, an optical disc, a magneto-optical disc, magnetic tape, or a Universal Serial Bus (USB) drive or a combination of two or more of these. The storage 608 may include removable or non-removable (or fixed) media, where appropriate. The storage 608 may be internal or external to computer system 600, where appropriate. In particular embodiments, the storage 608 is non-volatile, solid-state memory. In particular embodiments, the storage 608 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 608 taking any suitable physical form. The storage 608 may include one or more storage control units facilitating communication between processor 606 and storage 608, where appropriate. Where appropriate, the storage 608 may include one or more storages 608. Although this disclosure describes and illustrates particular storage, this disclosure contemplates any suitable storage.

In particular embodiments, the I/O Interface 610 includes hardware, software, or both, providing one or more interfaces for communication between computer system 600 and one or more I/O devices. The computer system 600 may include one or more of these I/O devices, where appropriate. One or more of these I/O devices may enable communication between a person (i.e., a user) and computer system 600. As an example and not by way of limitation, an I/O device may include a keyboard, keypad, microphone, monitor, screen, display panel, mouse, printer, scanner, speaker, still camera, stylus, tablet, touch screen, trackball, video camera, another suitable I/O device or a combination of two or more of these. An I/O device may include one or more sensors. Where appropriate, the I/O Interface 610 may include one or more device or software drivers enabling processor 606 to drive one or more of these I/O devices. The I/O interface 610 may include one or more I/O interfaces 610, where appropriate. Although this disclosure describes and illustrates a particular I/O interface, this disclosure contemplates any suitable I/O interface or combination of I/O interfaces.

In particular embodiments, communication interface 612 includes hardware, software, or both providing one or more interfaces for communication (such as, for example, packet-based communication) between computer system 600 and one or more other computer systems 600 or one or more networks 614. As an example and not by way of limitation, communication interface 612 may include a network interface controller (NIC) or network adapter for communicating with an Ethernet or any other wire-based network or a wireless NIC (WNIC) or wireless adapter for communicating with a wireless network, such as a Wi-Fi network. This disclosure contemplates any suitable network 614 and any suitable communication interface 612 for the network 614. As an example and not by way of limitation, the network 614 may include one or more of an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 600 may communicate with a wireless PAN (WPAN) (such as, for example, a Bluetooth® WPAN), a WI-FI network, a WI-MAX network, a cellular telephone network (such as, for example, a Global System for Mobile Communications (GSM) network), or any other suitable wireless network or a combination of two or more of these. Computer system 600 may include any suitable communication interface 612 for any of these networks, where appropriate. Communication interface 612 may include one or more communication interfaces 612, where appropriate. Although this disclosure describes and illustrates a particular communication interface implementations, this disclosure contemplates any suitable communication interface implementation.

The computer system 602 may also include a bus. The bus may include hardware, software, or both and may communicatively couple the components of the computer system 600 to each other. As an example and not by way of limitation, the bus may include an Accelerated Graphics Port (AGP) or any other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local bus (VLB), or another suitable bus or a combination of two or more of these buses. The bus may include one or more buses, where appropriate. Although this disclosure describes and illustrates a particular bus, this disclosure contemplates any suitable bus or interconnect.

Herein, a computer-readable non-transitory storage medium or media may include one or more semiconductor-based or other types of integrated circuits (ICs) (e.g., field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

All of the disclosed methods and procedures described in this disclosure can be implemented using one or more computer programs or components. These components may be provided as a series of computer instructions on any conventional computer readable medium or machine readable medium, including volatile and non-volatile memory, such as RAM, ROM, flash memory, magnetic or optical disks, optical memory, or other storage media. The instructions may be provided as software or firmware, and may be implemented in whole or in part in hardware components such as ASICs, FPGAs, DSPs, or any other similar devices. The instructions may be configured to be executed by one or more processors, which when executing the series of computer instructions, performs or facilitates the performance of all or part of the disclosed methods and procedures.

It should be understood that various changes and modifications to the examples described here will be apparent to those skilled in the art. Such changes and modifications can be made without departing from the spirit and scope of the present subject matter and without diminishing its intended advantages. It is therefore intended that such changes and modifications be covered by the appended claims.

1. A method comprising:
receiving a first image depicting a first dog;
identifying, with a first model, a first breed for the first dog based on the first image;
determining, with a second model, a first body condition for the first dog based on the first image;
generating, with a third model, a second image depicting the first dog with a second body condition different from the first body condition;
labeling the first image with indications of the breed and the first body condition;
labeling the second image with indications of the breed and the second body condition; and
training the second model using the first and second images.

2. The method of claim 1, wherein the second model corresponds to the first breed and is selected from among a plurality of models corresponding to a plurality of breeds.

3. The method of claim 1, wherein the third model corresponds to the first breed and at least one target body condition, the at least one target body condition including the second body condition.

4. The method of claim 3, wherein the third model corresponds to a plurality of target body conditions, and wherein generating the second image further includes generating multiple images depicting the first dog with at least a subset of the plurality of target body conditions.

5. The method of claim 1, wherein the first image and the second image are side-view images of the first dog.

6. The method of claim 1, further comprising:
receiving a third image depicting a second dog and labeled with an indication of a second breed; and
generating, based on the first image and the third image, a fourth image depicting a dog with a mixed breed of the first breed and the second breed.

7. The method of claim 1, wherein the first and second body conditions include at least one of an underweight condition, an ideal weight condition, an overweight condition, and an obese condition.

8. The method of claim 7, wherein the second body condition is the ideal weight condition, and wherein the method further comprises providing the second image to an owner of the first dog.

9. The method of claim 1, wherein the third model is a deep learning model.

10. The method of claim 9, wherein the third model is a neural network model with dense mapping.

11. The method of claim 1, wherein the second model includes a neural network model and a classifier model.

12. A system comprising:
a processor; and
a memory storing instructions which, when executed by the processor, cause the processor to:
receive a first image depicting a first dog;
identify, with a first model, a first breed for the first dog based on the first image;
determine, with a second model, a first body condition for the first dog based on the first image;
generate, with a third model, a second image depicting the first dog with a second body condition different from the first body condition;
label the first image with indications of the breed and the first body condition;
label the second image with indications of the breed and the second body condition; and
train the second model using the first and second images.

13. The system of claim 12, wherein the second model corresponds to the first breed and is selected from among a plurality of models corresponding to a plurality of breeds.

14. The system of claim 12, wherein the third model corresponds to the first breed and at least one target body condition, the at least one target body condition including the second body condition.

15. The system of claim 14, wherein the third model corresponds to a plurality of target body conditions, and wherein generating the second image further includes generating multiple images depicting the first dog with at least a subset of the plurality of target body conditions.

16. The system of claim 12, wherein the first image and the second image are side-view images of the first dog.

17. The system of claim 12, wherein the instructions further cause the processor to:
receive a third image depicting a second dog and labeled with an indication of a second breed; and
generate, based on the first image and the third image, a fourth image depicting a dog with a mixed breed of the first breed and the second breed.

18. The system of claim 12, wherein the first and second body conditions include at least one of an underweight condition, an ideal weight condition, an overweight condition, and an obese condition.

19. The system of claim 18, wherein the second body condition is the ideal weight condition, and wherein the instructions further cause the processor to providing the second image to an owner of the first dog.

20. The system of claim 12, wherein the third model is a deep learning model.
